(19) United States
(12) Patent Application Publication
(10) Pub. No.: US 2011/0171240 A1
(43) Pub. Date: Jul. 14, 2011

(54) USE OF CCR9, CCL25/TECK, AND INTEGRIN ALPHA4 IN DIAGNOSIS AND TREATMENT OF MELANOMA METASTASIS IN THE SMALL INTESTINE

(76) Inventors: Dave S. B. Hoon, Los Angeles, CA (US); Farin Amersi, Los Angeles, CA (US)

(21) Appl. No.: 12/982,670

(22) Filed: Dec. 30, 2010

Related U.S. Application Data
(62) Division of application No. 11/829,507, filed on Jul. 27, 2007.

(51) Int. Cl. A61K 39/395; A61P 35/04; A61K 31/713

(52) U.S. Cl. 424/172.1; 514/44 A

(57) ABSTRACT

The invention relates to methods for determining whether a melanoma will metastasize or has metastasized to the small intestine in a subject by detecting or quantifying the expression of the CCR9, CCL25/TECK, or integrin α4 gene. Also disclosed are methods for treating subjects so identified.

### PRIORITY CLAIM

This application is a divisional application of U.S. application Ser. No. 11/829,507, filed Jul. 27, 2007, the content of which is hereby incorporated herein by reference in its entirety.

### FIELD OF THE INVENTION

The present invention relates in general to cancer. More specifically, the invention relates to the use of CCR9 (chemokine (C-C motif) receptor 9), CCL25/TECK (chemokine (C-C motif) ligand 25/thymus expressed chemokine), and integrin α4 as markers for diagnosing and treating melanoma metastasis to the small intestine.

### BACKGROUND OF THE INVENTION

Cutaneous melanoma continues to be a growing problem, as the incidence of malignant melanoma continues to increase 3-8% per year over the last several decades, faster than that of other malignancies.¹ Melanoma now accounts for 5% of all cancers diagnosed, and, according to the American Cancer Society, an estimated 62,190 cases of invasive melanoma were diagnosed in the United States in 2006. For patients with AJCC stage 1V disease, treatment options remain limited, and the prognosis is poor with a 5-year survival rate of approximately 10%. Melanoma frequently metastasizes to the gastrointestinal tract, with autopsies demonstrating disseminated disease in 50-60% of patients with AJCC stage 1V disease.²

It is known that peri-tumoral lymphatic vessels facilitate metastases to regional draining lymph nodes and the development of liver metastases from primary cutaneous melanoma requires tumor cell metastasis into the blood stream. However, melanoma demonstrates an unusual predilection to metastasize to the small bowel.^{3,4} The underlying mechanism for this is unknown. Small bowel metastases from other solid tumors are unusual when compared to the incidence of liver and colonic metastases, and this infrequent occurrence is even more surprising given that the small bowel comprises at least 75% of the entire length of the gastrointestinal tract.^{5,6} In the largest series reported in the literature of melanoma patients with metastases to the gastrointestinal tract, lesions were found primarily in the small bowel, and were less commonly seen in the stomach, colon, and rectum.⁷ The pathogenesis of this propensity for site-specific small bowel metastases by cutaneous melanoma is an enigma. Diagnosing and managing patients with intestinal metastases is often difficult due to the insidious nature of the disease. Most patients initially have non-specific symptoms, but may present later with advanced disease, causing gastrointestinal bleeding or obstruction where palliative surgery is the only option, unlike patients who present with early stage disease, where curative surgical resection with wide local excision of the primary lesion and lymphadenectomy is associated with improved survival.⁸ It has become increasingly apparent that tumor growth and organ predilection of metastases involves multiple complex interactions in the tumor microenvironment, whereby metastases establish at specific organs only if microenvironment requirements are met.^{9,10}

The phenomenon of seed and soil events for metastasis has been discussed for decades; however, preferential metastasis to specific organs is still not well understood. Some preferential metastases to certain sites, such as bone marrow, lung, and liver are primarily related to vascular drainage pattern, vicinity of original tumor, and supportive tissue microenvironments for metastasis.⁹⁻¹² Chemokine receptors and their corresponding ligands constitute a family of structurally related proteins that have been implicated in mediating tumor cell invasion and organ-specific trafficking of tumor cells leading to metastases.^{13,14} It is known that orchestration of immune events at specific organ sites is highly regulated by the chemokine-ligand axis (Sallusto F, Mackay C R, Lanzavecchia A: The role of chemokine receptors in primary, effector, and memory immune responses. Annu Rev Immunol 18:593-620, 2000). With activation of the chemokine-ligand during development of metastasis, tumor cells that express a chemokine receptor migrate along a chemokine gradient, allowing them to move to specific sites having higher concentrations of the chemokine (Sallusto F, Mackay C R, Lanzavecchia A: The role of chemokine receptors in primary, effector, and memory immune responses. Annu Rev Immunol 18:593-620, 2000).

### SUMMARY OF THE INVENTION

This invention relates to methods for diagnosis and treatment of melanoma metastasis in the small intestine based on the expression levels of the CCR9, CCL25/TECK, and integrin α4 genes.

In one aspect, the invention features a method of determining whether a melanoma will metastasize or has metastasized to the small bowel in a subject. One method of the invention comprises the steps of (1) providing a tissue sample of a melanoma primary tumor or a melanoma lymph node or skin metastasis, or a body fluid sample from a subject suffering from melanoma; and (2) determining the expression level of the CCR9 or integrin α4 gene in the tissue or body fluid sample. If the expression level of the CCR9 or integrin α4 gene in the tissue or body fluid sample is higher than a control level (e.g., the expression level of the CCR9 or integrin α4 gene in a corresponding tissue or body fluid sample from a normal person), the melanoma likely will metastasize or has metastasized to the small bowel.

Another method of the invention comprises the steps of (1) providing a body fluid sample from a subject suffering from melanoma, and (2) determining the expression level of the CCL25/TECK gene in the sample. If the expression level of the CCL25/TECK gene in the sample is higher than a control level (e.g., the expression level of the CCL25/TECK gene in a corresponding body fluid sample from a normal person), the melanoma likely will metastasize or has metastasized to the small bowel. In some embodiments, the CCR9 gene is expressed in the melanoma; in other embodiments, the CCR9 gene is not expressed in the melanoma.

The melanoma primary tumor or melanoma lymph node or skin metastasis tissue sample may be a PEAT (paraffin-embedded archival tissue), frozen, or fresh tissue sample. The body fluid sample may be a blood, serum, plasma, or bone marrow fluid sample. The expression level of the CCR9, integrin α4, or CCL25/TECK gene may be determined by qRT (quantitative reverse transcription polymerase chain reaction) or an antibody to the CCR9, integrin α4, or CCL25/TECK protein.

In another aspect, the invention features a method of inhibiting gene expression or protein-protein interaction in a subject. The method comprises the steps of (1) identifying a subject in which a melanoma will metastasize or has metastasized to the small bowel according to the method of the invention; and (2) contacting the subject with an agent that reduces the expression level of the CCR9, integrin α4, or CCL25/TECK gene, or blocks the interaction between the CCR9 protein and the CCL25/TECK protein. This method may be used to inhibit melanoma metastasis to the small bowel. The agent may be a CCR9, integrin α4, or CCL25/TECK siRNA (short interfering mRNA) that reduces the expression level of the CCR9, integrin α4, or CCL25/TECK gene; a monoclonal or polyclonal antibody to the CCR9 or CCL25/TECK protein that blocks the interaction between the CCR9 protein and the CCL25/TECK protein; or a CCR9 antagonist that blocks the interaction between the CCR9 protein and the CCL25/TECK protein.

The above-mentioned and other features of this invention and the manner of obtaining and using them will become more apparent, and will be best understood, by reference to the following description, taken in conjunction with the accompanying drawings. These drawings depict only typical embodiments of the invention and do not therefore limit its scope.

### BRIEF DESCRIPTION OF THE FIGURES

FIG. 1. CCR9 expression in melanoma cell lines. (A) CCR9 expression in melanoma cell lines derived from small bowel metastases. (B) No CCR9 expression in melanoma cell lines derived from melanoma metastases to visceral organs. Results are mean±SD.

FIG. 2. FACS analysis of CCR9 on melanoma cells. Flow cytometry detection of CCR9 expression on melanoma cell lines derived from small bowel metastases. Representative histograms are shown of two cell lines. (A) Positive control; (B) KJ liver metastatic cell line; (C) ML small bowel metastatic cell line; and (D) MK small bowel metastatic cell line.

FIG. 3. CCR9 expression in metastatic small bowel PEAT tissues. CCR9 mRNA expression by melanoma metastases to the small bowel assessed by qRT.

FIG. 4. Representative IHC staining for CCR9 expression. Representative IHC staining for CCR9 expression in melanoma small bowel metastases specimens demonstrating strong immunoreactivity (A1 and B1). Representative IHC staining of negative controls for small bowel metastases (A2 and B2). Metastatic melanoma to lung (C1) and liver (D1) demonstrating no immunostaining of CCR9. Representative IHC staining of negative controls for lung melanoma metastasis (C2) and liver melanoma metastasis (D2).

FIG. 5. CCR9 functional analysis on melanoma cell lines. Cell migration of two representative small bowel metastatic melanoma cell lines MP and MG (A). Stimulation with CCL25 (100 ng/ml) significantly increased the number of migrating (MP and MG) cells (both p<0.001) as determined by an invasion assay. □ No treatment; ▪ CCL25/CCR9. Two representative small bowel metastatic melanoma cell lines MP and MG (B). Addition of the anti-CCR9 antibody (1 μg/ml) resulted in a significant decrease in the number of cells that invaded across the Matrigel matrix insert in response to CCL25 (p<0.002 and p<0.004, respectively). □ No treatment; [symbol] CCL25/CCR9; ▪ CCL25+anti-CCR9 Ab.

FIG. 6. CCR9 siRNA transfection. qRT analysis of representative small bowel-derived metastatic melanoma cell lines ML (A) and MP (B) was performed after CCR9 siRNA and control siRNA transfection. After siRNA treatment, a significant decrease in CCR9 expression was seen in ML (p=0.002) cells and MP (p=0.004) cells. Cell migration assay of two representative small bowel metastatic melanoma cell lines ML (C) and MP (D) after CCR9 siRNA transfection following stimulation with CCL25. There was a significant decrease in the ability of transfected ML (C) and MP (D) cells to migrate in response to CCL25 (p<0.004 and p<0.01, respectively).

### DETAILED DESCRIPTION OF THE INVENTION

Chemokine receptor expression has been shown to be upregulated in many types of cancers, including melanoma, lung, breast, colon, and ovarian cancer.¹⁵⁻¹⁸ CXCR4 expression has been shown in multiple cancers of epithelial, hematopoietic, and mesenchymal origin, and CXCL12, the only known ligand for CXCR4, has been found at specific sites of metastases in breast, melanoma, colorectal, and ovarian cancer.¹⁹⁻²³ The propensity of certain tumors to develop site-specific metastases, such as gastric and colorectal cancer to the lung and liver, may be secondary to the vascular drainage patterns of these tumors, and the ability of endothelial cells in the vascular beds of these organs to express specific adhesion molecules that can trap circulating tumor cells. However, the propensity of melanoma metastases to develop in small bowel may relate to the “seed and soil phenomenon”, rather than dissemination of cancer cells preferentially through the circulation. Based on this evidence, which suggests that chemokines play a significant role in tumor cell trafficking and the development of organ-specific metastases, it was hypothesized that a potential “homing” chemoattractive relation may explain the mechanism by which melanoma preferentially metastasizes to the small bowel. The unusual physiology of cutaneous melanomas is that the tumor can originate at any anatomical site on the skin, whereas other types of solid tumors occur at specific organ sites.

Thymus expressed chemokine (TECK) or CCL25, a CC chemokine expressed predominantly in thymus and epithelium of the small intestine, has been shown to mediate chemotaxis of CCR9-bearing T-cells.^{24,25} A number of studies have shown selective expression of CCR9 on small bowel infiltrating T-cells, as well as intra-epithelial and lamina propria lymphocytes of the small bowel.²⁶⁻²⁸ Recent studies have shown more evidence of this site-specific immunity by demonstrating that, in patients with inflammatory bowel disease (IBD) affecting the small bowel, there are increased numbers of CCR9(+) lymphocytes circulating in peripheral blood.²⁹ This suggests that CCR9 may play a role in the pathogenesis of immune-mediated small bowel disorders.

The invention is based at least in part upon the unexpected discovery that cutaneous melanoma cells express CCR9 and respond to CCL25 of the small bowel, facilitating preferential metastasis from the primary lesion or draining lymph nodes to the small bowel. Accordingly, the invention provides diagnostic methods for determining whether a melanoma will metastasize or has metastasized to the small intestine in a subject.

As used herein, a “subject” refers to a human or animal, including all mammals such as primates (particularly higher primates), sheep, dog, rodents (e.g., mouse or rat), guinea pig, goat, pig, cat, rabbit, and cow. In a preferred embodiment, the subject is a human. In another embodiment, the subject is an experimental animal or animal suitable as a disease model.

A method of the invention involves obtaining a biological sample from a subject. A biological sample from a subject may be a tissue sample such as a biopsy specimen sample, a normal or benign tissue sample, a cancer or tumor sample, a freshly prepared tissue sample, a frozen tissue sample, a PEAT sample, a primary cancer or tumor sample, or a metastasis sample. Alternatively, a biological sample may be a sample of a body fluid. The term “body fluid” refers to any body fluid in which cells (e.g., cancer cells) may be present, including, without limitation, blood, serum, plasma, bone marrow, cerebral spinal fluid, peritoneal/pleural fluid, lymph fluid, ascite, serous fluid, sputum, lacrimal fluid, stool, and urine. Tissue and body fluid samples can be obtained from a subject using any of the methods known in the art.

The expression levels of genes in a biological sample are analyzed. “Gene expression” is a process where a gene is transcribed into an mRNA, which in turn is translated into a protein. Gene expression can be detected and quantified at the mRNA or protein level using a number of means well known in the art. To detect mRNAs or measure mRNA levels, cells in biological samples (e.g., tissues and body fluids) can be lysed and the mRNA in the lysates or in RNA purified or semi-purified from the lysates detected or quantified by any of a variety of methods familiar to those in the art. Such methods include, without limitation, hybridization assays using detectably labeled gene-specific DNA or RNA probes and quantitative or semi-quantitative RT-PCR (e.g., real-time PCR) methodologies using appropriate gene-specific oligonucleotide primers. Alternatively, quantitative or semi-quantitative in situ hybridization assays can be carried out using, for example, unlysed tissues or cell suspensions, and detectably (e.g., fluorescently or enzyme-) labeled DNA or RNA probes. Additional methods for quantifying mRNA levels include RNA protection assay (RPA), cDNA and oligonucleotide microarrays, and colorimetric probe based assays.

Methods for detecting proteins or measuring protein levels in biological samples are also known in the art. Many such methods employ antibodies (e.g., monoclonal or polyclonal antibodies) that bind specifically to target proteins. In such assays, an antibody itself or a secondary antibody that binds to it can be detectably labeled. Alternatively, the antibody can be conjugated with biotin, and detectably labeled avidin (a polypeptide that binds to biotin) can be used to detect the presence of the biotinylated antibody. Combinations of these approaches (including “multi-layer sandwich” assays) familiar to those in the art can be used to enhance the sensitivity of the methodologies. Some of these protein-measuring assays (e.g., ELISA or Western blot) can be applied to body fluids or to lysates of test cells, and others (e.g., immunohistological methods or fluorescence flow cytometry) applied to unlysed tissues or cell suspensions. Methods of measuring the amount of a label depend on the nature of the label and are known in the art. Appropriate labels include, without limitation, radionuclides (e.g., ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Other applicable assays include quantitative immunoprecipitation or complement fixation assays.

To practice the diagnostic methods of the invention, a melanoma primary tumor sample, a melanoma lymph node or skin metastasis sample, or a body fluid sample is obtained from a subject who suffers from melanoma. The expression level of the CCR9, integrin α4, or CCL25/TECK gene in the sample is then determined and compared to a control level. A control level may be the expression level of the CCR9, integrin α4, or CCL25/TECK gene in a corresponding (e.g., obtained from the same body location) tissue or body fluid sample from a normal subject. If the expression level of the CCR9, integrin α4, or CCL25/TECK gene in the test sample is higher than the control level, the melanoma likely will metastasize or has metastasized to the small bowel in the test subject.

In another aspect, the invention provides treatment methods for inhibiting melanoma metastasis to the small intestine in a subject who suffers from melanoma. A subject to be treated may be identified in the judgment of the subject or a health care professional, and can be subjective (e.g., opinion) or objective (e.g., measurable by a test or diagnostic method). According to the diagnostic methods described above, the melanoma likely will metastasize or has metastasized to the small intestine in the subject.

To treat a subject, an effective amount of an agent that reduces the expression level of the CCR9, CCL25/TECK, or integrin α4 gene, or inhibits the interaction between the CCR9 protein and the CCL25/TECK protein is administered to the subject. The expression level of a gene may be reduced, e.g., by inhibiting the transcription from DNA to mRNA or the translation from mRNA to protein. Alternatively, the expression level of a gene may be reduced by preventing mRNA or protein from performing their normal functions. For example, the mRNA may be degraded through anti-sense RNA, ribozyme, or siRNA; the protein may be blocked by a monoclonal or polyclonal antibody, or an antagonist. The agent may be administered in combination with other compounds or radiotherapy for melanoma.

The term “treatment” is defined as administration of a substance to a subject with the purpose to cure, alleviate, relieve, remedy, prevent, or ameliorate a disorder, symptoms of the disorder, a disease state secondary to the disorder, or predisposition toward the disorder. An “effective amount” is an amount of a compound that is capable of producing a medically desirable result in a treated subject. The medically desirable result may be objective (i.e., measurable by some test or marker) or subjective (i.e., subject gives an indication of or feels an effect).

Polynucleotides (i.e., anti-sense nucleic acid molecules, ribozymes, and siRNAs) can be delivered to target cells by, for example, the use of polymeric, biodegradable microparticle or microcapsule devices known in the art. Another way to achieve uptake of nucleic acid is using liposomes, prepared by standard methods. The polynucleotides can be incorporated alone into these delivery vehicles or co-incorporated with tissue-specific or tumor-specific antibodies. Alternatively, one can prepare a molecular conjugate composed of a polynucleotide attached to poly-L-lysine by electrostatic or covalent forces. Poly-L-lysine binds to a ligand that can bind to a receptor on target cells. “Naked DNA” (i.e., without a delivery vehicle) can also be delivered to an intramuscular, intradermal, or subcutaneous site. A preferred dosage for administration of a polynucleotide is from approximately 10⁶ to 10¹² copies of the polynucleotide molecule.

For treatment of melanoma, a compound is preferably delivered directly to tumor cells, e.g., to a tumor or a tumor bed following surgical excision of the tumor, in order to treat any remaining tumor cells. For prevention of cancer invasion and metastases, the compound can be administered to, for example, a subject that has not yet developed detectable invasion and metastases but is found to have increased expression of the CCR9, CCL25/TECK, or integrin α4 gene.

The compounds of the invention can be incorporated into pharmaceutical compositions. Such compositions typically include the compounds and pharmaceutically acceptable carriers. “Pharmaceutically acceptable carriers” include solvents, dispersion media, coatings, antibacterial and antifungal agents, isotonic and absorption delaying agents, and the like, compatible with pharmaceutical administration.

A pharmaceutical composition is formulated to be compatible with its intended route of administration. See, e.g., U.S. Pat. No. 6,756,196. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

In one embodiment, the compounds are prepared with carriers that will protect the compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of an active compound calculated to produce the desired therapeutic effect in association with the required pharmaceutical carrier.

The dosage required for treating a subject depends on the choice of the route of administration, the nature of the formulation, the nature of the subject's illness, the subject's size, weight, surface area, age, and sex, other drugs being administered, and the judgment of the attending physician. Suitable dosages are in the range of 0.01-100.0 mg/kg. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

The following example is intended to illustrate, but not to limit, the scope of the invention. While such example is typical of those that might be used, other procedures known to those skilled in the art may alternatively be utilized. Indeed, those of ordinary skill in the art can readily envision and produce further embodiments, based on the teachings herein, without undue experimentation.

### Example

### Activation of CCR9/CCL25 Expression Mediates Metastasis of Melanoma to the Small Intestine

Specific chemokines and their respective receptors have been shown to facilitate tumor-cell metastasis to specific distant organs. Melanoma has a distinct pattern of metastasis to the gastrointestinal tract; melanoma cells preferentially target the submucosa of the small bowel, rather than colon, stomach, or rectum. The underlying pathogenic mechanism for this is unknown. Human cutaneous melanoma is the most common cause of metastases in the small bowel, where CCL25, the ligand for chemokine receptor CCR9, is selectively expressed. This site-specific metastasis by melanoma cells may relate to the “seed and soil” phenomenon involving the small bowel. Here, CCR9 expression is demonstrated in 88 of 102 metastatic melanoma specimens from the small bowel, 7 of 8 melanoma cell lines derived from metastases in the small bowel, and 0 of 96 metastatic melanoma specimens from other sites. CCR9 expression was also common in primary melanomas that metastasized to the small bowel (p<0.05). In melanoma cell lines, CCR9 expression was correlated with cell migration in response to CCL25. The CCL25-induced migratory response was inhibited by anti-CCR9 antibody and by transfection of melanoma cells with short interfering mRNA for CCR9. These findings demonstrate that functionally active expression of CCR9 on melanoma cells facilitates migration of these cells specifically to the small bowel. Identification of the CCR9-CCL25 axis as a mechanism for site-specific metastasis explains the high incidence of small bowel metastases in patients with advanced melanoma. This finding is a demonstration of organ-specific metastasis, independent of location of primary tumor or vascular drainage pattern.

### Results

### Melanoma Cells Express CCR9

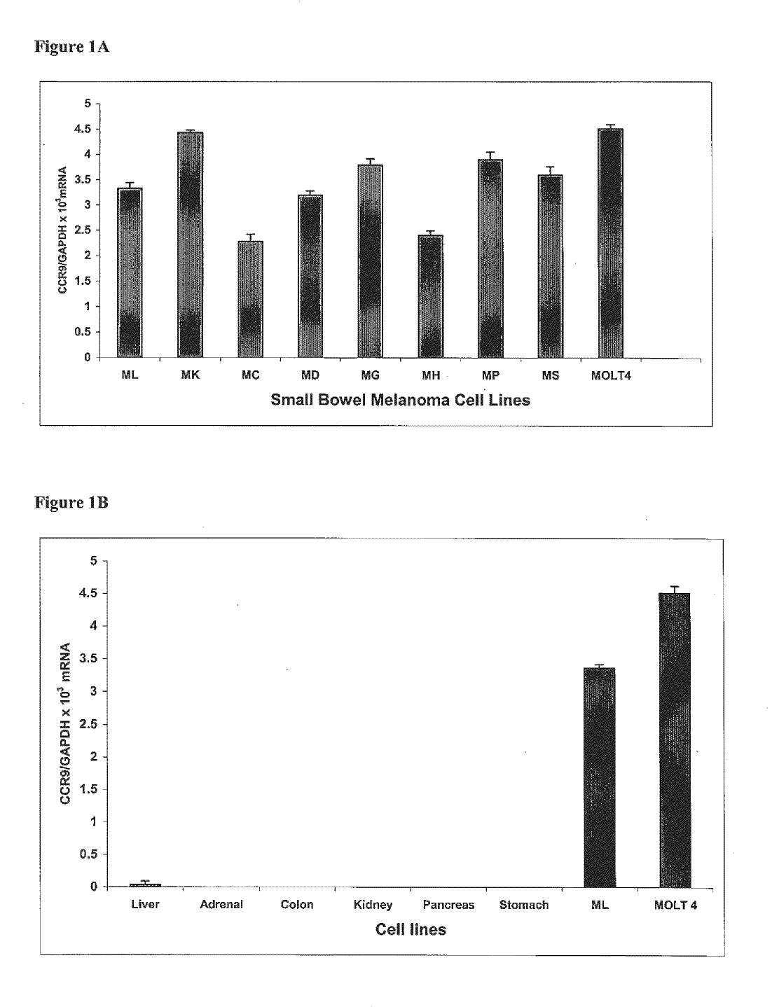

To determine if CCR9/CCL25 interactions play a role in melanoma metastases to the small bowel, CCR9 mRNA expression levels were assessed by qRT in 23 established metastatic melanoma cell lines. Of the 23 cell lines examined, 7 of 8 (87%) of the melanoma cell lines derived from small bowel metastases were positive for CCR9 expression (FIG. 1A). All seven positive cell lines expressed the CCR9 gene. The CCR9 mRNA copy levels were normalized with GAPDH mRNA expression levels to determine the relative expression of the gene. CCR9:GAPDH mRNA levels ranged from 2.28 to 4.74×10³. The fifteen remaining cell lines from melanoma metastases to other distant sites (liver=4, colon=2, stomach=1, adrenal=2, lung=3, pancreas=1, kidney=2) showed no expression of CCR9 (FIG. 1B). Five of the seven small bowel metastasis lines that had high CCR9 mRNA expression (3.33 to 4.74×10³; ML, MG, MP, MS, MK) were used for subsequent studies.

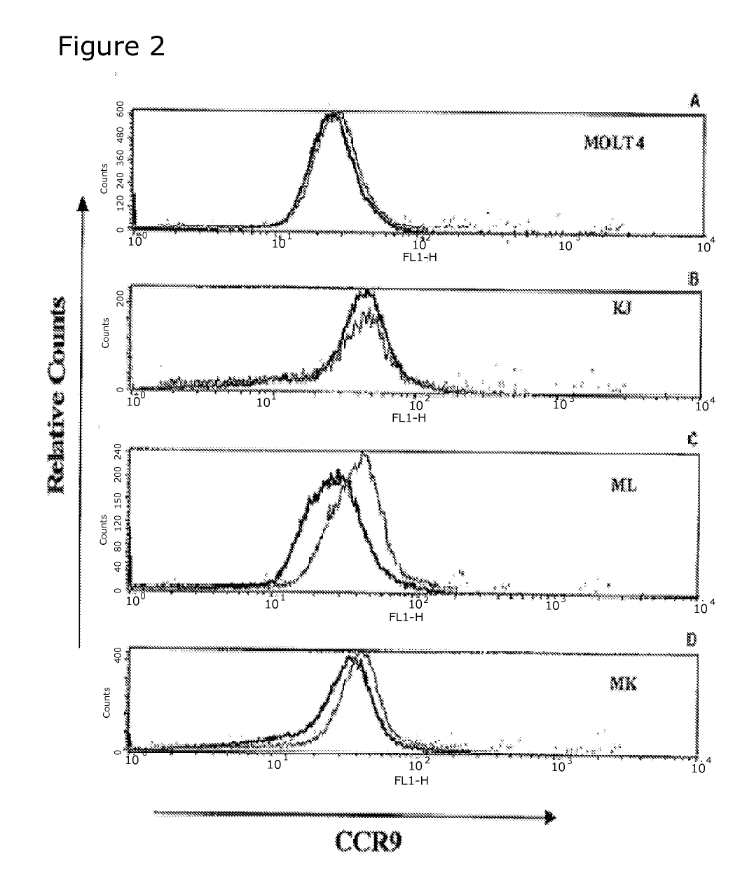

To validate CCR9 mRNA expression levels, metastatic melanoma cell lines were examined for CCR9 expression by flow cytometry. Cell lines derived from small bowel intestinal metastases were used to determine expression of CCR9. As shown in FIG. 2, CCR9 surface receptor was detected on melanoma cells isolated from small bowel metastases, and was in the same range as was found in that of the human T cell leukemia line MOLT-4, a known positive control for CCR9.³⁰ CCR9 was not detected in a control melanoma cell line derived from a metastasis to the liver.

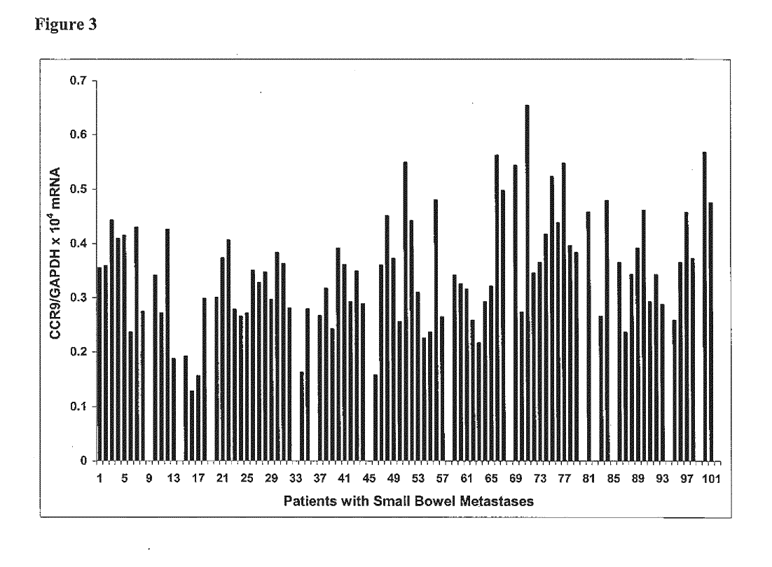

Because the findings showed a role for CCR9 in vitro, it was sought to determine if these findings would correlate in vivo in metastases from visceral organs, as the microenvironment in vivo may be different from cell populations in the in vitro environment. By qRT, the expression level of CCR9 in specimens obtained from 198 melanoma patients who underwent surgical resection of metastases at distant organ sites was assessed. Each paraffin-embedded archival tissue (PEAT) specimen was from metastatic melanoma in the small bowel, liver, gallbladder, pancreas, adrenal glands, stomach, colon, or lung. The CCR9 mRNA copy levels were normalized with GAPDH mRNA expression levels to determine the relative expression and for comparison of different patient specimens, as previously described.²³ CCR9 gene expression was detected in 88 of 102 (86%) small bowel metastases (FIG. 3). For 72 of the 88 patients (82%), the small bowel was the only site of metastatic disease found during surgery. All 14 patients whose small bowel metastases did not express CCR9 had multiple liver metastases and metastatic disease in the colon, spleen, kidney, or adrenal glands. Similarly, specimens (PEAT) obtained from patients who had undergone surgical resection for metastases to liver (n=19), kidney (n=5), lung (n=14), gallbladder (n=9), pancreas (n=8), adrenal (n=7), stomach (n=18), and colon (n=16) demonstrated no expression of the CCR9 gene. The mRNA quality of all specimens was verified, as previously described.³¹ These studies validated the specificity of CCR9 expression for the development of small bowel metastases.

In order to validate the presence of CCR9 on cutaneous melanoma cells, PEAT specimens from 23 patients who had previously undergone wide local excision of a primary cutaneous melanoma were also assessed. The 14 truncal lesions, 4 head and neck lesions, and 5 upper extremity lesions had a mean Breslow thickness of 3.57±0.54 mm. Eleven of 23 (48%) specimens demonstrated CCR9 expression. Seven of the 11 tumors (64%) were from patients who subsequently developed small bowel metastases; the remaining four specimens were from patients who have not developed any regional lymph node or distant metastases to date. CCR9:GAPDH mRNA levels ranged from 0.14 to 0.43×10².

### CCL25/TECK

Expression of CCL25 (TECK) in metastatic melanoma from the small bowel versus other sites was investigated by qRT analysis of PEAT specimens. The quality of the specimens was once again verified through analysis of GAPDH mRNA. The range of CCL25/GAPDH mRNA levels was higher in the 88 small bowel specimens that had previously been shown to express CCR9 than in the 14 small bowel specimens that did not demonstrate expression of CCR9 (1.71×10² to 3.41×10² vs. 0.97×10² to 1.27×10², respectively). In addition, what other studies have shown was confirmed: no expression of CCL25 in metastatic melanoma specimens from the liver, kidney, lung, gallbladder, pancreas, adrenal, stomach, and colon, when compared to normal small bowel, which was used as a positive control. This data demonstrates that expression of CCL25, the only known ligand for CCR9, is upregulated in melanoma patients who develop small bowel metastases.

The expression level of CCR9 was also investigated in metastatic tissue from regional lymph nodes of 22 patients who had undergone lymph node dissection for nodal disease at the time of excision of the primary tumor, and subsequently developed small bowel metastases. The regional lymph nodes are the most common site of early stage metastasis from primary cutaneous melanoma, and there is in vitro evidence of CCR9 expression in nodal metastases.³² PEAT specimens from 10 of 22 (45%) of the patients demonstrated CCR9 expression (p<0.05). None of the nodal specimens expressed CCL25. It is likely that other chemokines, such as CCL21 and CXCL12, are involved in supporting nodal metastasis.³³

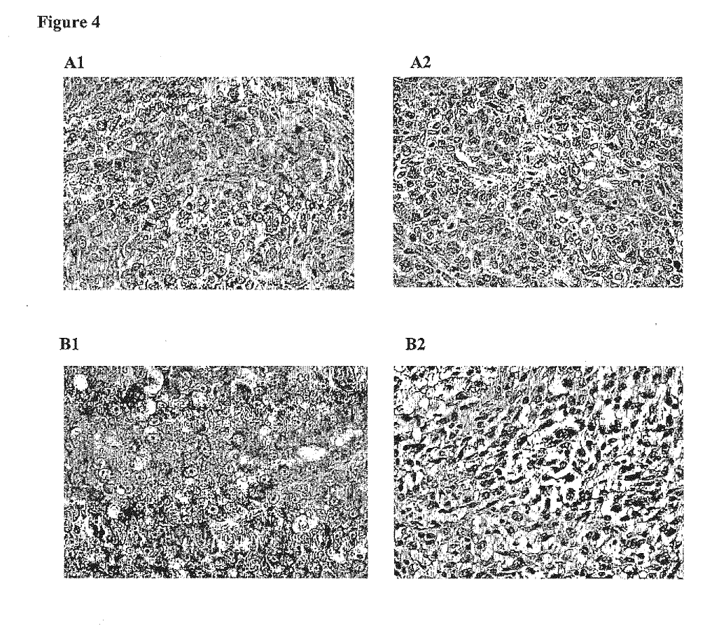

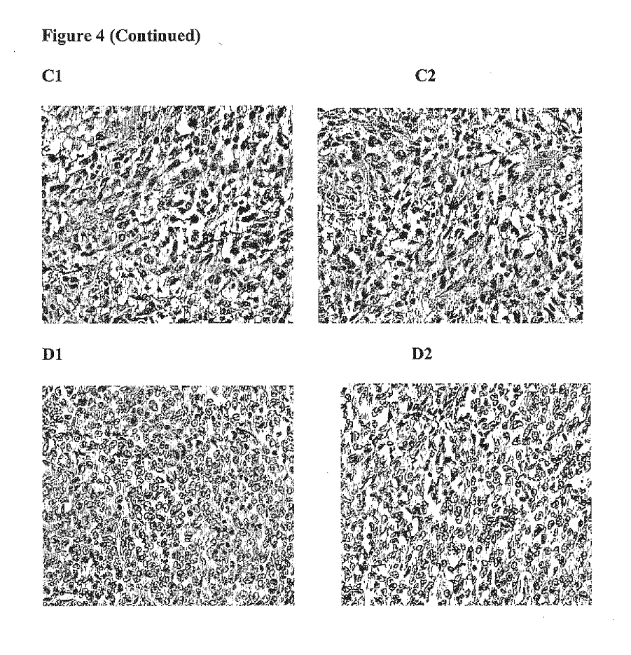

Furthermore, the presence of CCR9 protein expression in small bowel melanoma metastases was examined by immunohistochemistry (IHC) with a monoclonal mouse anti-human CCR9 antibody. CCR9 expression was confirmed in PEAT specimens that had been analyzed in the qRT analysis as being positive. The intensity of staining was variable (FIGS. 4A1, 4A2, 4B1 and 4B2). No staining was seen in PEAT specimens of metastatic melanomas from other organ sites (FIGS. 4C1, 4D1, and 4D2). These findings also correlated with CCR9 gene expression analysis by qRT.

### Migratory and Chemoinvasive Responses to CCL25

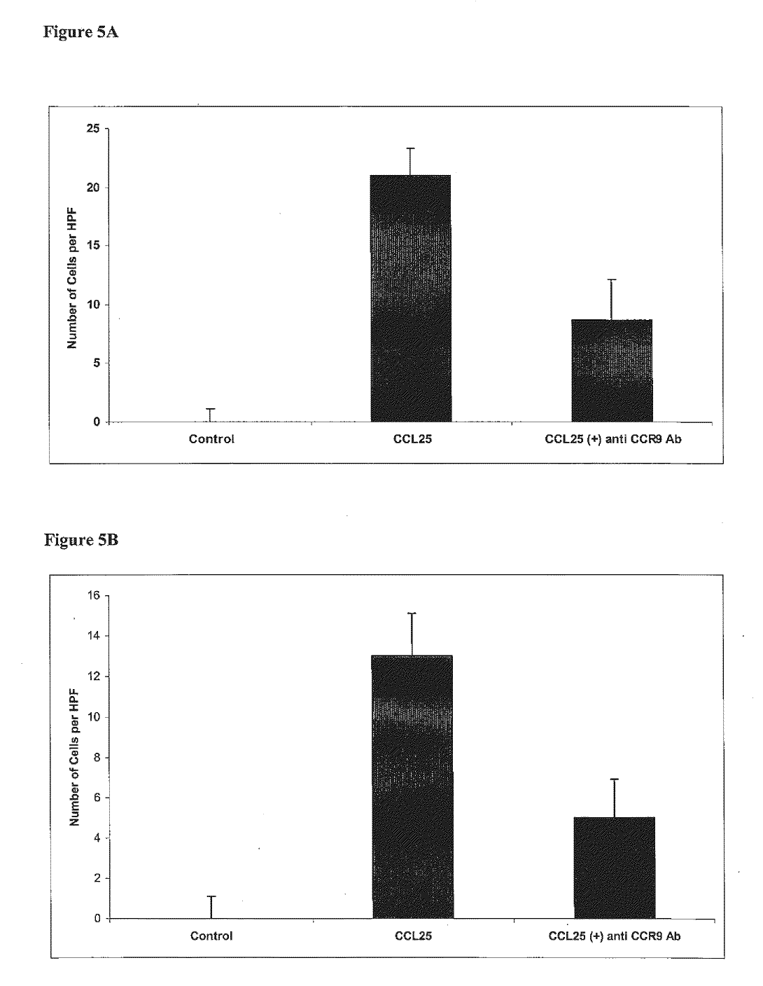

Chemotaxis and tumor invasion are important components in the series of steps whereby metastasis to specific organs occurs. Therefore, the chemotactic response of melanoma cells to CCL25, the ligand for CCR9, was assessed in a cell migration assay. Four of the small bowel cell lines, ML, MP, MG, and MK that had demonstrated expression of CCR9 by qRT, were assessed for CCR9/CCL25 responses. The functional significance of CCR9 was demonstrated by the ability of CCL25 to induce migration of melanoma cells in these four cell lines. The number of melanoma cells that migrated in response to CCL25 was significantly higher than that of untreated controls (p<0.001; FIG. 5A). These findings demonstrate a correlation between increased CCR9 mRNA expression and an increase in the number of melanoma cells migrating in response to CCL25.

A Matrigel chemoinvasion assay demonstrated that melanoma cells which expressed CCR9 were more invasive when stimulated with CCL25 (p<0.001). Pre-treatment of the melanoma cell lines MP and MG with anti-CCR9 antibody, significantly inhibited (p<0.002 and p<0.004, respectively) the ability of melanoma cells to migrate across the Matrigel matrix in response to CCL25 (FIG. 5B). These findings demonstrate that activation of CCR9 by CCL25 on melanoma cells can promote migration and invasion.

Effect of CCR9 siRNA

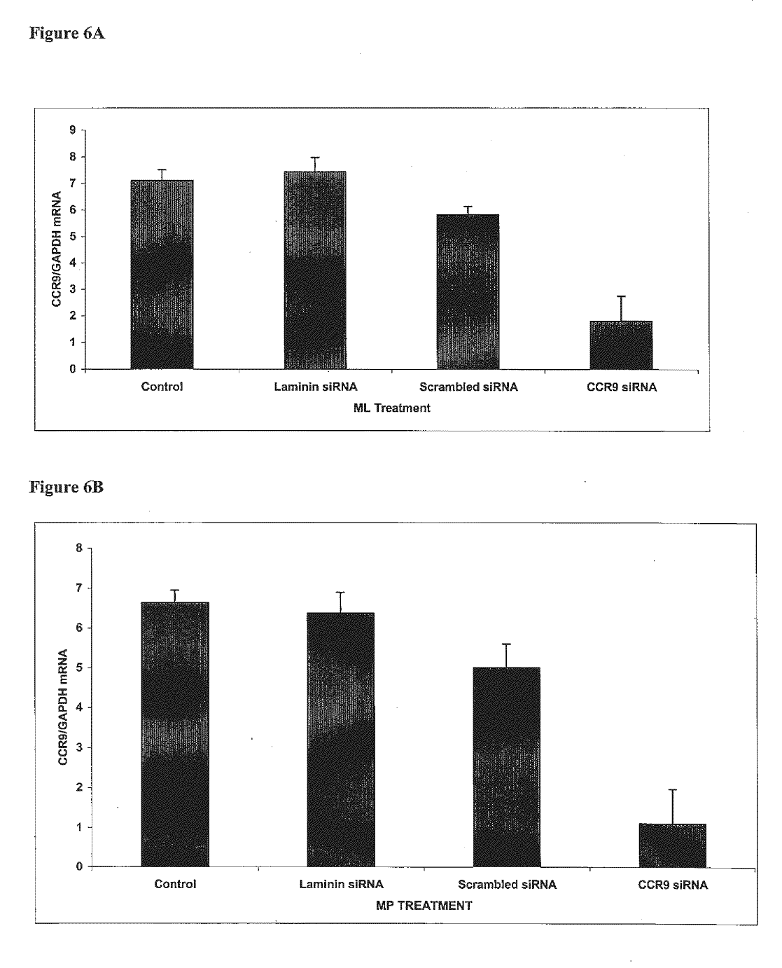

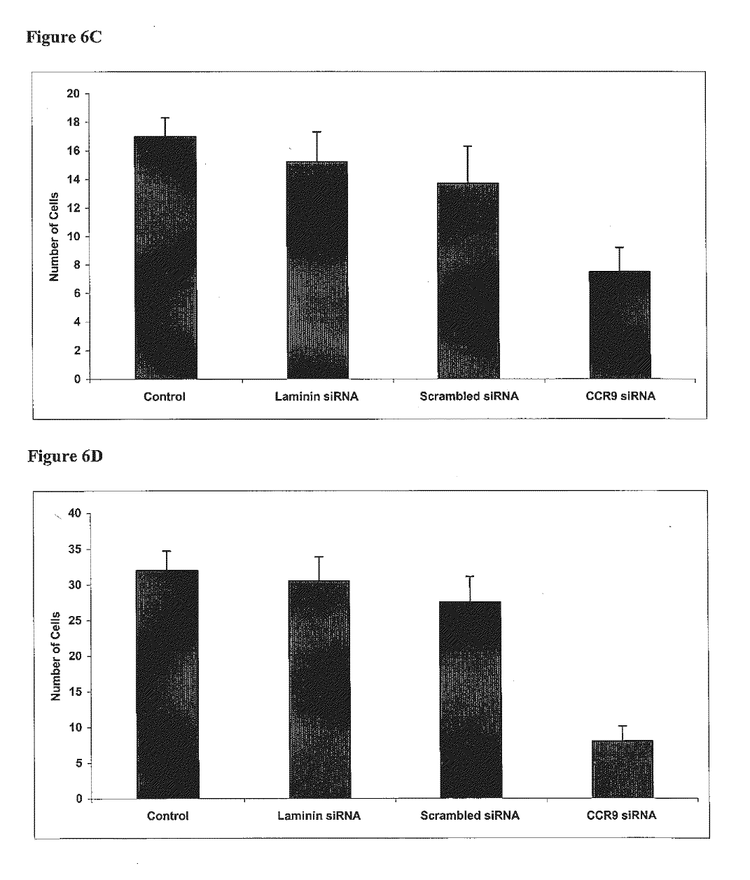

Short interfering RNA (siRNA) was used in vitro on cells to downregulate CCR9 mRNA expression, and evaluate functional response of melanoma cells to CCL25. The CCR9(+) small bowel melanoma cell lines (MP and ML) were selected as representative metastatic lines and transfected with CCR9 siRNA. As demonstrated by qRT analysis (FIGS. 6A and 6B), transfection of MP and ML cells with CCR9 siRNA decreased expression of CCR9 mRNA by 76% (p=0.004) in MP cells and by 87% (p=0.002) in ML cells. The efficiency of transfection was assessed by comparison to scrambled siRNA and positive (laminin) control cells.

ML and MP cells transfected with CCR9 siRNA were then assessed for their migratory responses to CCL25. The functional significance of CCR9 downregulation by CCR9 siRNA was demonstrated by the presence of CCL25 to induce migration of melanoma cells (FIGS. 6C and 6D). The number of melanoma cells that migrated in response to CCL25 was significantly lower than that of scramble siRNA-transfected control cells (p<0.004 and p<0.01, respectively). The migratory responses were impaired by 76% and 63%, respectively, for the small bowel melanoma lines ML and MP.

### Discussion

Evidence from many studies suggests that chemokines and their receptors regulate the growth and migration of various cancer.^{34,35} For example, in breast cancer the chemokine receptor CXCR4 may be predominantly involved in metastasis to the bone marrow, whereas chemokine receptor CCR7 has been linked to preferential nodal metastasis.³⁶ Melanoma is anomalous because, unlike breast cancer metastasis, which usually targets the bones, liver, or lung, and unlike colon cancer which usually targets the liver, melanoma is relatively nondiscriminating; it may target almost any part of the body. Although its most frequent destination is the skin or lymph nodes, melanoma has a uniquely high (26-58%) rate of metastasis to the gastrointestinal tract. This is a unique metastasis site pattern for any human solid tumor.

Site-specific metastasis begins when cells from a primary solid malignancy are shed into vascular or lymphatic channels. The “seed and soil” phenomenon does not fully explain the specificity of tumor-specific metastasis. Vascular drainage patterns and vicinity of the primary tumor has a significant influence on most solid tumors. The event of CTC adhesion and growth sequence does not explain fully why tumor cells may migrate only to a particular organ site. Previously, it was demonstrated that melanoma patients of different stages of disease have CTC which are related to disease outcome.^{37,38}

Specific chemokine-ligand axes are a promising answer to the puzzling questions that surround organ-specific metastasis.^{39,40} It was found that the CCR9-CCL25 axis may play an important role in the preferential homing of melanoma cells to the small bowel, where CCL25 is expressed in abundance. Recently, it has been demonstrated that variable expression of chemokine receptors in melanoma cell lines, a finding that reflects the well-known heterogeneity of this cancer and might explain its wide range of metastatic targets.^{41,42}

Studies have implicated CCR9(+) peripheral T-cells in metastasis to the small bowel.⁴³⁻⁴⁶ CCL25, which is selectively expressed only in the thymus and small bowel, has been found to activate specific subsets of T-cells that have a homing mechanism for the gut mucosa.⁴⁷ Integrins α4 and β7 also play an important role in mucosal homing, and these adhesion molecules have been identified in gut-associated lymphoid tissue (GALT) and in T-cells in the lamina propria of the small bowel.^{45,46} It is speculated that coexpression of CCR9 and integrins might characterize circulating intestinal memory T-cells that preferentially migrate to the small bowel. FACS and IHC analysis of melanoma cell lines derived from small bowel metastases revealed high expression of α4 in addition to CCR9. β7 was not detected, but its absence might have been an artifact of the in vitro setting or the quality of the antibody available.

CCR9-CCL25 axis interactions may play a pivotal role in anti-apoptosis via multiple signaling pathways involving Akt and glycogen synthase kinase 3β.⁴⁸ Papakadis et al. reported a five-fold increase in CCR9(+) T-cells in the blood of patients with inflammation of the small bowel but not the colon, which suggests the involvement of these T-cells in the pathogenesis of immune-mediated disease of the small bowel.²⁸

This study is the first to demonstrate preferential metastasis of CCR9-expressing melanoma cells to the small bowel. CCR9 expression was identified in metastatic melanoma tissue from the small bowel but not other organ sites; parallel in vitro assays demonstrated that CCR9 expression on cells derived from small bowel metastases increased cell migration in response to CCL25. Interestingly, CCR9 expression was also demonstrated in primary melanomas from patients who subsequently developed small bowel metastases.

These findings implicate the CCR9-CCL25 axis in preferential metastasis of melanoma to the small bowel. In the study of almost 200 specimens from visceral metastases of melanoma, CCR9 expression was identified only in specimens from the small bowel; similarly, when normal tissue from the same sites was assessed, CCL25 was only identified in specimens from the small bowel. Upregulation of CCR9 expression in melanoma cells may be triggered by changes in the microenvironment of the skin and/or small bowel, which predispose melanoma cells to target and colonize the small bowel. Further studies will determine the regulatory mechanism of CCR9 expression by primary cutaneous melanoma and events involved in establishment of small bowel metastasis. CCR9 antagonists could merit investigation as a therapy to prevent metastasis of CCR9(+) melanoma cells.

### Methods

### Melanoma Cell Lines and Paraffin-Embedded Tissues

Twenty-three cell lines established from metastatic melanoma tumors of patients at the John Wayne Cancer Institute were assessed. Human T-cell leukemia cell line MOLT 4 (American Type Culture Collection, Rockville, Md.), which has been described previously to express CCR9, was used as a positive control.³⁰ Cell lines were maintained in RPMI 1640 supplemented medium (Gibco, Carlsbad, Calif.), supplemented with heat-inactivated 10% fetal bovine serum, 1% penicillin G, and streptomycin (100 units/ml) at 37° C. with 5% CO₂, as previously described.²³

Patients who had undergone surgical resection for visceral metastases of melanoma, were selected from the John Wayne Cancer Institute, Santa Monica, Calif. (JWCI) melanoma database by the database manager. All patients were treated at either JWCI or the Sydney Cancer Center, Royal Prince Alfred Hospital, Camperdown, Australia from 1996 through 2005. Tumor specimens were obtained from primary melanomas (n=5 AJCC stage HA, n=11 AJCC stage IIB, n=7 AJCC Stage IIC), regional lymph nodes (n=22), and distant sites (n=198) including small bowel, liver, colon, stomach, lung, pancreas, gallbladder, adrenal, and kidney that were routinely fixed with 10% buffered formalin and embedded in paraffin following tissue processing. All PEAT blocks were obtained from the Department of Surgical Pathology of each respective institution, only after approval of the Institutional Review Board (IRB) was obtained. Normal small bowel PEAT specimens were used as control tissues.

### RNA Isolation

Total cellular RNA from melanoma cell lines was extracted using Tri-Reagent (Molecular Research Center, Cincinnati, Ohio), as previously described.³¹ For PEAT, 10 sections of 10 μm thick tissues were cut from each block. Deparaffinized tissue sections were digested using proteinase K, and RNA was extracted using a modified protocol of the RNAWiz Isolation Kit (Ambion, Austin, Tex.), as previously described.³¹ The RNA was quantified and assessed for purity by UV spectrophotometry and by the RIBOGreen detection assay (Molecular Probes, Eugene, Oreg.), as previously described, using a defined standard operation procedure.³¹ All RNA samples were treated with Turbo DNAase (Ambion, Austin, Tex.) to remove residual genomic DNA contamination in the RNA solutions prior to performing reverse transcription of total RNA. Respective control reactions were run to determine DNA-free status of samples.

### Primers and Probes

The primer and probe sequences were designed using the Oligo 6 Primer Analysis Software (National Biomedical Systems, Plymouth, Minn.), and verified as previously described.²² In order to avoid the potential amplification of contaminating genomic DNA, the primers were designed such that each product covered at least one exon-intron-exon region. The primers and FRET probe sequences used were as follows: CCR9 (110 bp): 5′-GCCTGAGCAGGGAGATTAT-3′ (SEQ ID NO:1) (forward), 5′-GGAGCAGACAGACGGTG-3′ (SEQ ID NO:2) (reverse), and 5′-FAM-CAAGTGCCACTCAACAGAACAAGC-BHQ-1-3′ (SEQ ID NO:3) (FRET probe). CCL25 (131 bp): 5′-CCATCAGCAGCAGTAAGAGG-3′ (SEQ ID NO:4) (forward), 5′-CTGTAGGGCGACGGTTTTAT-3′ (SEQ ID NO:5) (reverse), and 5′-FAM-CTGTGAGCCGGCTCATTTCTG-BHQ-1-3′ (SEQ ID NO:6) (FRET probe). Glyceraldehyde-3-phoshate dehydrogenase. (GAPDH; 136 bp): 5′GGGTGTGAACCATGAGAAGT-3′ (SEQ ID NO:7). (forward), 5′·GACTGTGGTCATGAGTCCT-3′ (SEQ ID NO:8) (reverse), and 5′-FAM-CAGCAATGCCTCCTGCACCACCAA-BHQ-1-3′ (SEQ ID NO:9) (FRET probe).

Quantitative RT-PCR (qRT) Assays

Reverse transcription of total RNA was performed using Moloney murine leukemia virus RT (Promega, Madison, Wis.) with Oligo dT (GeneLink, Hawthorne, N.Y.) and random hexamers (Roche, Indianapolis, Ind., USA) for priming, as previously described for PEAT sections and cell lines.²³ The quantitative real-time RT-PCR (qRT) assay was performed with the ABI Real-Time PCR System (Applied Biosystems, Foster City, Calif.) where cDNA from 250 ng of total RNA was used for each reaction. The PCR reaction mixture consisted of 0.25 μm of each primer, 0.25 μm FRET probe, 12.5 μL of Universal master mix (Applied Biosystems, Foster City, Calif.), and 6.75 μL water to a final volume of 20 μL. For CCR9 analysis, samples were amplified at 45 cycles of denaturation at 95° C. for 1 min, annealing at 58° C. for 1 min, and extension at 72° C. for 1 min; CCL25: 40 cycles at 95° C. for 1 min, 55° C. for 1 min, and 72° C. for 1 min; GAPDH: 45 cycles at 95° C. for 1 min, 55° C. for 1 min, and 72° C. for 1 min. Each sample was assayed in triplicate, and appropriate positive and negative tissues, reagents, and assay controls were included in each assay. Verification of mRNA integrity from samples was assessed.

### Cell Migration and Invasion Assays

Migration and invasion studies were performed on cell lines using a modified Boyden transwell chamber chemotaxis assay.⁴² The cell migration assay was performed using a 6.5-mm diameter transwell double chamber with 8-μm pore filters (HTS Transwell-24 System; Corning, Acton, Mass.). The lower surfaces of the insert membranes were precoated with Laminin (20 μg/ml) for 2 h at room temperature. Human recombinant CCL25 was obtained from Peprotech (Rocky Hill, N.J.) and added to the lower wells of the Boyden chamber with serum-free medium and 0.1% albumin. Melanoma cells (10⁴) were seeded in the upper chamber and incubated overnight at 37° C. in 5% CO₂. After incubation, the cells in the upper chamber that had not migrated were removed using cotton swabs, and the cells that had migrated at the bottom of the membrane were fixed in 100% ethanol, washed with phosphate buffer solution, and then stained with 1% crystal violet. The number of cells in four randomly selected fields at 200× and 400× magnification were counted as previously described.²³

For the Matrigel chemoinvasion assays, the modified Boyden chamber system was used, and laminin was coated on the underside of the inserts, and a layer of Matrigel (BD Biosciences, Franklin Lakes, N.J.) was placed within the insert. Melanoma cells were treated with an unlabeled mouse anti-human CCR9 antibody (1.0 μg/ml) (R&D Systems, Minneapolis, Minn.) 2 h prior to assay performance. Human recombinant CCL25 was added to the lower wells of the Boyden chamber with serum-free medium and 0.1% albumin. Melanoma cells (10⁴) were seeded in the upper chamber and incubated for 48 h at 37° C. in 5% CO₂. Invading cells that had migrated at the bottom of the membrane were fixed in 100% ethanol, washed with phosphate buffer solution, and then stained with 1% crystal violet. Cells were evaluated as described above.

### Flow Cytometry

Melanoma cells (10⁵) were washed in PBS (pH 7.0), trypsinized, and treated with 1.0 μg of Fc Block (BD PharMingen, San Diego, Calif.) per 10⁵ cells for 15 minutes at 37° C. The melanoma cells were then incubated with fluorescein-conjugated mouse monoclonal IgG₂ₐ anti-human CCR9 antibody (1:100 dilution; Santa Cruz Biotechnology, Santa Cruz, Calif.) or the isotype matched control conjugated mouse IgG₂ₐ antibody (BD PharMingen, San Diego, Calif.) at 4° C. for 60 min. The cells were then labeled with goat anti-mouse IgG-FITC for 60 min. An anti-MHC class 1 antibody (BD Pharmingen) was used as a positive control. The labeled cells were then fixed in propridium iodide solution, and analyzed using a FACScan flow cytometer (FACSCalibur, Becton Dickinson, Mountain View, Calif.) and Cell Quest analysis software (Becton Dickinson).

### Short Interfering RNA Assay

To determine the role of CCR9 gene expression on melanoma cells, a CCR9 siRNA assay was developed. The melanoma cell lines, ML and MP, were used as representative metastatic melanoma small bowel cell lines. Human CCR9 siRNA duplexes, a scrambled siRNA duplex, and an siRNA positive control were developed (Dharmacon Research Inc, Lafayette, Colo.). Melanoma cells (10⁵) were seeded into 6-well culture plates, and maintained in RPMI medium. After the cells became confluent, the medium was changed to serum-free medium for 6 h. Melanoma cells were then transfected for 8 h using 200 uM siRNA duplexes with lipofectamine 2000 (Invitrogen, Carlsbad, Calif.), as previously described.⁴⁹ After transfection, the medium was changed to full-growth medium for 48 h. The medium was then changed to serum-free medium, after which cells were harvested for analysis. All experiments for each of the cell lines ML and MP were done in triplicates.

### Immunohistochemistry

Expression of CCR9 was confirmed by IHC on 5 μm thick sections of PEAT small bowel metastases, as well as other visceral metastases. The sections were incubated overnight at 37° C., deparaffinized in xylene, and treated with citrate buffer for heat-induced epitope recovery, pH 6.0 (Diagnostic BioSystems Inc., Pleasanton, Calif.) at 95° C. for 20 min, and then cooled to room temperature for 20 min. CSAII Kit (Dakocytomation, Carpinteria, Calif.) was then used for the staining process. The sections were incubated overnight at 4° C. with a monoclonal mouse anti-human CCR9 antibody (1:200 dilution; R & D Systems). Negative control slides were incubated with normal mouse IgG (Santa Cruz Biotechnology, Santa Cruz, Calif.) under similar conditions. After 24 hrs, sections were developed using the Vector VIP substrate kit (Dakocytomation), and examined at 400× magnification under a phase contrast light microscope.

### Statistical Analysis

Data are presented as mean±SE, and statistical analysis of the data was performed using a two-tailed Student's t test or an unpaired Mann-Whitney U Test. Differences were considered statistically significant at a p value of <0.05. All analyses were performed using SAS (SAS/STAT User's Guide, version 8; SAS Institute Inc, Cary, N.C.).

### REFERENCES

1. Thompson, J. F., Scolyer, R. A. & Kefford, R. F. Cutaneous melanoma. Lancet 365, 687-701 (2005).
2. Dasgupta, T. & Brasfield, R. Metastatic Melanoma. A Clinicopathological Study. Cancer 17, 1323-39 (1964).
3. Agrawal, S., Yao, T. J. & Coit, D. G. Surgery for melanoma metastatic to the gastrointestinal tract. Ann Surg Oncol 6, 336-44 (1999).
4. Reintgen, D. S., Thompson, W., Garbutt, J. & Seigler, H. F. Radiologic, endoscopic, and surgical considerations of melanoma metastatic to the gastrointestinal tract. Surgery 95, 635-9 (1984).
5. Ashley, S. W. & Wells, S. A., Jr. Tumors of the small intestine. Semin Oncol 15, 116-28 (1988).
6. Schuchter, L. M., Green, R. & Fraker, D. Primary and metastatic diseases in malignant melanoma of the gastrointestinal tract. Curr Opin Oncol 12, 181-5 (2000).
7. Ollila, D. W., Essner, R., Wanek, L. A. & Morton, D. L. Surgical resection for melanoma metastatic to the gastrointestinal tract. Arch Surg 131, 975-9; 979-80 (1996).
8. Morton, D. L. et al. Sentinel-node biopsy or nodal observation in melanoma. N Engl J Med 355, 1307-17 (2006).
9. Kaplan, R. N., Rafii, S. & Lyden, D. Preparing the “soil”: the premetastatic niche. Cancer Res 66, 11089-93 (2006).
10. Witz, I. P. & Levy-Nissenbaum, O. The tumor microenvironment in the post-PAGET era. Cancer Lett 242, 1-10 (2006).
11. Nozawa, H. et al. Detection of cancer cells in mesenteric vein and peripheral vessels by measuring telomerase activity in patients with colorectal cancer. Surgery 134, 791-8 (2003).
12. Guise, T. A. et al. Basic mechanisms responsible for osteolytic and osteoblastic bone metastases. Clin Cancer Res 12, 6213s-6216s (2006).
13. Muller, A. et al. Involvement of chemokine receptors in breast cancer metastasis. Nature 410, 50-6 (2001).
14. Chambers, A. F., Groom, A. C. & MacDonald, I. C. Dissemination and growth of cancer cells in metastatic sites. Nat Rev Cancer 2, 563-72 (2002).
15. Kawada, K. et al. Pivotal role of CXCR3 in melanoma cell metastasis to lymph nodes. Cancer Res 64, 4010-7 (2004).
16. Darash-Yahana, M. et al. Role of high expression levels of CXCR4 in tumor growth, vascularization, and metastasis. Faseb J 18, 1240-2 (2004).
17. Scotton, C. J., Wilson, J. L., Milliken, D., Stamp, G. & Balkwill, F. R. Epithelial cancer cell migration: a role for chemokine receptors? Cancer Res 61, 4961-5 (2001).
18. Burger, M. et al. Functional expression of CXCR4 (CD184) on small-cell lung cancer cells mediates migration, integrin activation, and adhesion to stromal cells. Oncogene 22, 8093-101 (2003).
19. Su, Y. C. et al. Expression of CXCR4 is associated with axillary lymph node status in patients with early breast cancer. Breast 15, 533-9 (2006).
20. Zeelenberg, I. S., Ruuls-Van Stalle, L. & Roos, E. The chemokine receptor CXCR4 is required for outgrowth of colon carcinoma micrometastases. Cancer Res 63, 3833-9 (2003).
21. Scala, S. et al. Expression of CXCR4 predicts poor prognosis in patients with malignant melanoma. Clin Cancer Res 11, 1835-41 (2005).
22. Kim, J. et al. Chemokine receptor CXCR4 expression in colorectal cancer patients increases the risk for recurrence and for poor survival. J Clin Oncol 23, 2744-53 (2005).
23. Takeuchi, H. et al. CCL21 chemokine regulates chemokine receptor CCR7 bearing malignant melanoma cells. Clin Cancer Res 10, 2351-8 (2004).
24. Wurbel, M. A. et al. Mice lacking the CCR9 CC-chemokine receptor show a mild impairment of early T- and B-cell development and a reduction in T-cell receptor gammadelta(+) gut intraepithelial lymphocytes. Blood 98, 2626-32 (2001).
25. Zabel, B. A. et al. Human G protein-coupled receptor GPR-9-6/CC chemokine receptor 9 is selectively expressed on intestinal homing T lymphocytes, mucosal lymphocytes, and thymocytes and is required for thymus-expressed chemokine-mediated chemotaxis. J Exp Med 190, 1241-56 (1999).
26. Kunkel, E. J. et al. Lymphocyte CC chemokine receptor 9 and epithelial thymus-expressed chemokine (TECK) expression distinguish the small intestinal immune compartment: Epithelial expression of tissue-specific chemokines as an organizing principle in regional immunity. J Exp Med 192, 761-8 (2000).
27. Johansson-Lindbom, B. et al. Functional specialization of gut CD103+ dendritic cells in the regulation of tissue-selective T cell homing. J Exp Med 202, 1063-73 (2005).
28. Autschbach, F., Funke, B., Katzenmeier, M. & Gassler, N. Expression of chemokine receptors in normal and inflamed human intestine, tonsil, and liver—an immunohistochemical analysis with new monoclonal antibodies from the 8th international workshop and conference on human leucocyte differentiation antigens. Cell Immunol 236, 110-4 (2005).
29. Papadakis, K. A. et al. CCR9-positive lymphocytes and thymus-expressed chemokine distinguish small bowel from colonic Crohn's disease. Gastroenterology 121, 246-54 (2001).
30. Youn, B. S., Kim, C. H., Smith, F. O. & Broxmeyer, H. E. TECK, an efficacious chemoattractant for human thymocytes, uses GPR-9-6/CCR9 as a specific receptor. Blood 94, 2533-6 (1999).
31. Takeuchi, H. et al. Prognostic significance of molecular upstaging of paraffin-embedded sentinel lymph nodes in melanoma patients. J Clin Oncol 22, 2671-80 (2004).
32. Letsch, A. et al. Functional CCR9 expression is associated with small intestinal metastasis. J Invest Dermatol 122, 685-90 (2004).
33. Hoon, D. S. et al. Molecular mechanisms of metastasis. Cancer Metastasis Rev 25, 203-20 (2006).
34. Wang, J. et al. Chemokine receptor 7 activates phosphoinositide-3 kinase-mediated invasive and prosurvival pathways in head and neck cancer cells independent of EGFR. Oncogene 24, 5897-904 (2005).
35. Liang, Z. et al. Inhibition of breast cancer metastasis by selective synthetic polypeptide against CXCR4. Cancer Res 64, 4302-8 (2004).
36. Fidler, I. J. Seed and soil revisited: contribution of the organ microenvironment to cancer metastasis. Surg Oncol Clin N Am 10, 257-69, vii-viiii (2001).
37. Koyanagi, K. et al. Serial monitoring of circulating melanoma cells during neoadjuvant biochemotherapy for stage III melanoma: outcome prediction in a multicenter trial. J Clin Oncol 23, 8057-64 (2005).
38. Hoon, D. S. et al. Molecular markers in blood as surrogate prognostic indicators of melanoma recurrence. Cancer Res 60, 2253-7 (2000).
39. Kakinuma, T. & Hwang, S. T. Chemokines, chemokine receptors, and cancer metastasis. J Leukoc Biol 79, 639-51 (2006).
40. Murakami, T., Cardones, A. R. & Hwang, S. T. Chemokine receptors and melanoma metastasis. J Dermatol Sci 36, 71-8 (2004).
41. Seidl, H. et al. Profiles of chemokine receptors in melanocytic lesions: de novo expression of CXCR6 in melanoma. Hum Pathol (2007).
42. Mori, T. et al. Epigenetic up-regulation of C-C chemokine receptor 7 and C-X-C chemokine receptor 4 expression in melanoma cells. Cancer Res 65, 1800-7 (2005).
43. Mora, J. R. et al. Selective imprinting of gut-homing T cells by Peyer's patch dendritic cells. Nature 424, 88-93 (2003).
44. Johansson-Lindbom, B. et al. Selective generation of gut tropic T cells in gut-associated lymphoid tissue (GALT): requirement for GALT dendritic cells and adjuvant. J Exp Med 198, 963-9 (2003).
45. Svensson, M. et al. CCL25 mediates the localization of recently activated CD8alphabeta(+) lymphocytes to the small-intestinal mucosa. J Clin Invest 110, 1113-21 (2002).
46. Kunkel, E. J., Campbell, D. J. & Butcher, E. C. Chemokines in lymphocyte trafficking and intestinal immunity. Microcirculation 10, 313-23 (2003).
47. Uehara, S., Song, K., Farber, J. M. & Love, P. E. Characterization of CCR9 expression and CCL25/thymus-expressed chemokine responsiveness during T cell development: CD3(high)CD69+ thymocytes and gammadeltaTCR+thymocytes preferentially respond to CCL25. J Immunol 168, 134-42 (2002).
48. Youn, B. S. et al. Role of the CC chemokine receptor 9/TECK interaction in apoptosis. Apoptosis 7, 271-6 (2002).
49. Takeuchi, H. et al. X-Linked inhibitor of apoptosis protein expression level in colorectal cancer is regulated by hepatocyte growth factor/C-met pathway via Akt signaling. Clin Cancer Res 11, 7621-8 (2005).

The contents of all references cited herein are incorporated by reference in their entirety.

What is claimed is:

1. A method of inhibiting gene expression or protein-protein interaction in a subject to inhibit invasion and metastasis of melanoma cancer to the small bowel comprising:
identifying a subject in which a melanoma will metastasize or has metastasized to the small bowel;
administering to the subject an agent that (i) reduces the expression level of a chemokine C-C motif receptor 9 (CCR9), integrin α4, or chemokine C-C motif ligand 25/thymus expressed chemokine (CCL25/TECK) gene, or (ii) blocks the interaction between the CCR9 protein and the CCL25/TECK protein.

2. The method of claim 1, wherein the subject in which a melanoma will metastasize or has metastasized to the small bowel is identified according to a method comprising detecting whether CCR9 expression is present in a melanoma tumor tissue sample; and determining the melanoma has an increased likelihood to metastasize to the small bowel or has metastasized to the small bowel when CCR9 expression is present in the melanoma tumor tissue sample.

3. The method of claim 2, wherein the melanoma tumor tissue sample is a primary melanoma tumor, a melanoma metastatic lymph node or a melanoma skin metastasis.

4. The method of claim 2, wherein the agent is delivered directly to tumor cells present in the melanoma tumor tissue sample.

5. The method of claim 1, wherein the subject in which a melanoma will metastasize or has metastasized to the small bowel is identified according to a method comprising detecting whether CCL25/TECK expression is present in a body fluid sample obtained from the subject; and determining the melanoma has an increased likelihood to metastasize to the small bowel or has metastasized to the small bowel when CCL25/TECK expression is present in the body fluid sample.

6. The method of claim 1, wherein the agent is a CCR9, integrin α4, or CCL25/TECK siRNA (short interfering mRNA) that reduces the expression level of the CCR9, integrin α4, or CCL25/TECK gene.

7. The method of claim 1, wherein the agent is a monoclonal or polyclonal antibody to the protein that blocks the interaction between the CCR9 protein and the CCL25/TECK protein.

8. The method of claim 1, wherein the agent is an antagonist that blocks the interaction between the CCR9 protein and the CCL25/TECK protein.

9. A method of treating melanoma cancer by inhibiting gene expression or protein-protein interaction in a subject having melanoma cancer, comprising administering to the subject a pharmaceutical composition, the pharmaceutical composition comprising an agent that (i) reduces the expression level of the CCR9, integrin α4, or CCL25/TECK gene, or (ii) blocks the interaction between the CCR9 protein and the CCL25/TECK protein.

10. The method of claim 9, wherein the pharmaceutical composition is administered directly to melanoma tumor cells.

11. The method of claim 9, wherein the pharmaceutical composition is administered to a subject that suffers from melanoma cancer, but has not yet developed detectable metastases but is found to have increased expression of a CCR9, CCL25/TECK or integrin alpha 4 gene.

12. The method of claim 11, wherein the treatment results in prevention of cancer invasion and/or metastases.

13. The method of claim 9, wherein the agent is a CCR9, integrin α4, or CCL25/TECK siRNA (short interfering mRNA) that reduces the expression level of the CCR9, integrin α4, or CCL25 gene.

14. The method of claim 9, wherein the agent is a monoclonal or polyclonal antibody to the CCR9 or CCL25/TECK protein that blocks the interaction between the CCR9 protein and the CCL25/TECK protein.

15. The method of claim 9, wherein the agent is an antagonist that blocks the interaction between the CCR9 protein and the CCL25/TECK protein.


SEQUENCE LISTING (9 sequences)

SEQ ID NO: 1 - DNA, 19 bp, Artificial Sequence
  Synthetic oligonucleotide
gcctgagcagggagattat

SEQ ID NO: 2 - DNA, 17 bp, Artificial Sequence
  Synthetic oligonucleotide
ggagcagacagacggtg

SEQ ID NO: 3 - DNA, 24 bp, Artificial Sequence
  Synthetic oligonucleotide
caagtgccactcaacagaacaagc

SEQ ID NO: 4 - DNA, 20 bp, Artificial Sequence
  Synthetic oligonucleotide
ccatcagcagcagtaagagg

SEQ ID NO: 5 - DNA, 20 bp, Artificial Sequence
  Synthetic oligonucleotide
ctgtagggcgacggttttat

SEQ ID NO: 6 - DNA, 21 bp, Artificial Sequence
  Synthetic oligonucleotide
ctgtgagccggctcatttctg

SEQ ID NO: 7 - DNA, 20 bp, Artificial Sequence
  Synthetic oligonucleotide
gggtgtgaaccatgagaagt

SEQ ID NO: 8 - DNA, 19 bp, Artificial Sequence
  Synthetic oligonucleotide
gactgtggtcatgagtcct

SEQ ID NO: 9 - DNA, 24 bp, Artificial Sequence
  Synthetic oligonucleotide
cagcaatgcctcctgcaccaccaa